(19) United States
(12) Patent Application Publication
(10) Pub. No.: US 2004/0037866 A1
(43) Pub. Date: Feb. 26, 2004

(54) Composition and method for the treatment and prevention of adhesions

(76) Inventors: John N. Semertzides, Cincinnati, OH (US); Richard L. Grant, Cincinnati, OH (US)

Correspondence Address: FROST BROWN TODD, LLC, 2200 PNC CENTER, 201 E. FIFTH STREET, CINCINNATI, OH, 45202, US

(21) Appl. No.: 10/643,664

(22) Filed: Aug. 19, 2003

Related U.S. Application Data
(60) Provisional application No. 60/404,650, filed on Aug. 20, 2002.

(51) Int. Cl. A61F 2/00

(52) U.S. Cl. 424/423; 424/93.7

(57) ABSTRACT

Compositions and methods are described for the treatment and prevention of abdominal and thoracic adhesions as well as other adhesions using a cell-sustaining and surface-separating composition that nourishes and sustains grafted or present non-keratinizing (i.e. nonepidermal) epithelial cells. The composition is preferably a suspension of viable epithelial cells in a polymerizable, absorbable composition, such as fibrin glue, that will provide separation of the organ surfaces and nourishment to seeded and grafted cells.

### CROSS REFERENCE TO RELATED APPLICATION

This application claims priority from U.S. Provisional Patent Application Serial No. 60/404,650, Grant and Semertzides, filed Aug. 20, 2002, incorporated herein by reference.

### FIELD OF THE INVENTION

This invention generally relates to the prevention and treatment of adhesions. More particularly, this invention relates to a composition and method for the prevention and treatment of abdominal and thoracic adhesions as well as other adhesions, using a cell-sustaining and surface-separating composition that nourishes and sustains grafted or present non-keratinizing (i.e. non-epidermal) epithelial cells.

### BACKGROUND OF THE INVENTION

An adhesion is the abnormal union of separate tissue surfaces that often occurs during the healing process of injured tissues and organs. Adhesions may result after any trauma, such as a surgery or a wound, is sustained by the body. Damage to the epithelial or surface layer of the organ often causes these injuries. Injury can also be caused by manipulation of the tissue during surgery. The majority of soft tissues or organs have an epithelial layer or “skin” on their surface, such as the serosal layer of the intestine (visceral peritoneum) or parietal peritoneum of the abdominal wall. This layer is comprised of epithelial cells and is known as an epithelium. One function of the epithelial layer serves to separate the surfaces of the organs. When injury occurs, other cells, such as muscle cells, are exposed and the body forms scar tissue to close the wound. Scar tissue forms rapidly and in an unorganized manner, which often results in adjacent surfaces growing together resulting in adhesion. Adhesions are indiscriminate as they may form on organs ranging from the heart to female reproductive organs. The formation of adhesions creates serious medical problems because they often interfere with the proper functioning of an organ and may result in significant pain as well as the total loss of function in that organ.

Adhesions of the bowel involve some of the most serious problems. Since the small intestine alone has an average adult length of 9 to 12 feet in a healthy person, adhesions can be numerous, as moving loops of the small intestine are in constant contact with each other creating an environment conducive to the formation of adhesions. Another source of adhesion formation is between the parietal peritoneum, omentum, and intestines, as the intestines are completely encased by the visceral peritoneum. The omentum is a double layer of peritoneum attached to the stomach and draping over the small bowel in the abdominal cavity. Bowel adhesions can create severe abdominal pain and interfere with the digestive process, which can be life threatening.

Treatment methods for adhesions vary from organ to organ. While one form of adhesion treatment may work well on one specific organ, it may not work as well on another organ because organs differ in various ways such as in anatomy, function, and relative motion. For example, barrier materials or gels have proven to be ineffective when used on surfaces of organs in motion.

One method of treating adhesions is disclosed in U.S. Pat. Nos. 5,002,551, Linksy, issued Mar. 26, 1991, and 5,007,916, Linsky et al., issued Apr. 16, 1991 (the '551 and '916 patents). The method disclosed in these patents consists of the application of an absorbable adhesion barrier comprised of a knitted fabric of oxidized regenerated cellulose. The patents disclose that the material becomes a gel in less than three days to form a physical barrier. However, the art also teaches that the material is permeable, as defined by an open area of 12% to 20% and a high density (8 to 15 mg/cm²). Empirical evidence is used to support the need for the open area and high density. The high density of the fabric produces large amounts of acid as it is absorbed, which may kill some cells including the epithelial cells that are regenerating. Also, the material is only indicated for use in the pelvis.

U.S. Pat. No. 5,601,579, Semertzides, issued Feb. 11, 1997, relates to a strip of barrier material, such as oxidized regenerated cellulose, which is useful in preventing adhesions. The '579 patent teaches that this type of barrier material may be used when the potential for infection is low and the material is to be attached to the bowel using sutures. The '579 patent also teaches that a liquid protein, such as thrombin, may be applied to the bowel surface to prevent leakage of fibrin from the bowel. It is further taught that the strip of oxidized regenerated cellulose barrier material can form a gel barrier that is adequate for a total surface injury involving a bowel of 300 sq. in. or less. For larger injuries involving the bowel, healing may be more problematic since absorption of this material can take up to three months. The gel formed from oxidized regenerated cellulose can cause inflammation and with larger amounts of this material, healing may be delayed as it is sensitive to infection.

U.S. Pat. No. 5,605,938, Roufa et al., issued Feb. 25, 1997, relates to inhibiting scar tissue and adhesions using dextran sulfate. U.S. Pat. Nos. 5,900,245, Sawhney, issued May 4, 1999; 6,051,248, Sawhney et al., issued Apr. 18, 2000; and 6,352,710, Sawhney at al., issued Mar. 5, 2002, relate to the use of a tissue sealant known as FOCAL SEAL® manufactured by Focal, Inc. These patents teach the use of this sealant for the treatment of any medical condition which requires a coating or sealing layer, including barriers applied to prevent post-surgical adhesions by using the sealant to deliver drugs which are useful in the prevention of adhesions. However, it is noted that using the sealant to deliver antioxidants inhibits the reformation of the epithelial layer.

U.S. Pat. No. 6,258,124, Darois et al., issued Jul. 10, 2001, relates to another form of adhesion prevention directed to repair of inguinal hernias. This patent teaches the use of a barrier which has a porous mesh layer and a non-permeable barrier layer. The mesh layer promotes cell growth and allows for in-growth reinforcing the tissue. It also helps to prevent reoccurrence of the hernia.

The present invention provides for the use of proteins and more specifically fibrin glue in the prevention and treatment of adhesions.

### SUMMARY OF THE INVENTION

The present invention provides an adhesion treatment and prevention composition and method for using said composition. The composition comprises an absorbable, cell-sustaining and separating substance, such as a protein or polysaccharide. The composition is preferably a suspension of viable non-keratinizing epithelial cells for grafting on an injured surface to allow reformation of the epithelium. This may prevent further scar tissue from forming and also prevent the formation of adhesions. The composition and methods may be used to treat injury of both internal organs and the wall of the body cavity.

In accordance with the present invention, non-keratinizing epithelial cells are harvested and mixed with a protein to yield a composition 45. The amount of cells and protein used to create composition 45 is an effective amount of each, such that there is an effective amount of cells which is sufficient to facilitate the tissue regeneration process and that there is a sufficient amount of protein so as to serve as an effective medium for cell growth and nourishment. The protein may preferably be an adhesive or sealant such as fibrin glue, to assure that the organ does not leak and to secure the composition on the injured surface; however, other proteins or absorbable polymers may be used separately or in combination. Preferably, viable cells are mixed with the fibrin glue, which is a two-part liquid that is blended as it is applied to the surface allowing polymerization of the glue to start as the glue is being applied and for polymerization to be completed after being applied to the surface to yield the composition 45. Cells may be added to one or both of the two part liquids.

The compositions and methods of the present invention comprise the following elements for adhesion prevention:

a. Epithelial Seed Cells: Non-keratinizing epithelial seed cells should be applied or be present on the surface of the injured area to allow rapid re-growth or reformation of the epithelial layer. Non-keratinizing cells are cells that are not epidermal and do not form keratin, which is characteristically found in epidermal tissue such as skin or nails. Cells may be grafted to the surface of the injury in a number of methods but applying cells in a suspension is preferred.

b. Separation and Stability: The injured area should be kept separate from surrounding tissue until the epithelial layer can reform to provide a surface that will naturally not adhere to surrounding tissue surfaces. Separation and stability may be achieved using several methods including, but not limited to, covering with a protein or absorbable polymer such as a tissue sealant, tissue adhesive, absorbable fabric, mesh or strip.

c. Cell Nourishing: Most absorbable proteins and some polymers can provide nourishment for seed cells and also act to preserve the viability of cells when in colloidal suspension.

d. Aseptic: An internal injury should be covered with a nourishing protein or other material such as a polysaccharide to provide an environment for growing cells to repair the injury. However, this may also allow for pathogens to reproduce rapidly, possibly creating an abscess that will be a more serious complication than adhesions. For this reason, care needs to be taken to avoid leakage of bowel contents or contamination.

In the method of the present invention a composition comprising fibrin-based tissue sealant, glue or adhesive, and optionally viable, non-epidermal, epithelial cells, is applied to one or more injured internal surfaces so as to temporarily separate the injured area from other tissue and to protect, nourish and promote the regeneration of epithelial cells to reform an epithelial layer over the injured area.

More specifically, the present invention provides a method for the treatment and prevention of adhesions, comprising the steps of:

(a) surgically accessing an animal or human pelvis, abdomen, thorax, pericardium, spinal cord, dura, tendon, tendon sheath or tissues covered by an epithelial layer where adhesions have formed or may form;

(b) dividing one or more adhesions that may be present or conducting other surgery which creates an injured area;

(c) providing viable epithelial cells or assuring that viable epithelial cells are present in or surrounding said injured area; and

(d) applying an absorbable substance, protein or polymer in one or more layers over said viable epithelial cells and said injured area to stabilize and temporarily separate the injured area from the surrounding organ surfaces.

Finally, the present invention relates to a composition for the prevention or treatment of adhesions, adapted for application to an injured internal surface in the body, comprising:

(a) viable, non-epidermal, epithelial cells;

(b) an absorbable substance capable of maintaining the viability of said cells and that is at least partially suspending or covering said cells; and

(c) a means to temporarily separate the injured surface from surrounding tissue surfaces.

All patents and other documents noted in this application are incorporated by reference herein.

### BRIEF DESCRIPTION OF THE DRAWINGS

FIG. 1. is a front, internal view of a human peritoneal cavity with the intestines located therein.

FIG. 1a is an enlarged view of the intestines shown in FIG. 1.

FIG. 2 is an enlarged view of intestines with various adhesions.

FIG. 3 is an orthogonal cross-sectional view of small intestines shown in FIG. 2 to show the various tissue layers and an injury made if an adhesion was divided.

FIG. 4 is a cross-sectional view of the intestine shown in FIG. 3 and the adjacent abdominal wall with an adhesion between the two structures. The adhesion is extended and somewhat exaggerated to show what it would look like prior to being divided.

FIG. 5 is a cross-sectional view of the intestine and adjacent abdominal wall shown in FIG. 4 after division of the adhesion.

FIG. 6 is a cross-sectional view of the intestine, adjacent abdominal wall, and divided adhesion shown in FIG. 5 after application of the present invention composition of cells and protein to the injured surfaces of the intestine and abdominal wall.

FIGS. 7a, b and c is a sequence of close-up cross-sectional views of the intestine at the surface of the injury in FIG. 6, to show the reformation of the epithelial layer and seed cells suspended in the protein. FIG. 7a shows the composition in place in the injury immediately following application of the composition. FIG. 7b shows the composition after the composition is partially absorbed and a layer of epithelial cells is forming on the injured surface, perhaps one or two days after the surgery. FIG. 7c shows the epithelial layer partially reformed and the presence of released cells in the space adjacent to the injury.

FIG. 8 is a cross-sectional view of the intestine, adjacent abdominal wall, divided adhesion, and covering layers of cells and protein shown in FIG. 6 where the layer of protein and cells are stabilized by a strip of absorbable polymer, mesh, or fabric that is sutured in place on the intestine. No additional strip is required on the abdominal wall since the relative motion is less and there is better blood supply resulting in faster healing.

FIG. 9 is a cross-sectional view of the intestine, adjacent abdominal wall, divided adhesion, and covering layer of cells and protein shown in FIG. 6 where the layer of protein and cells are stabilized by a strip of absorbable polymer, mesh, or fabric wrapping around the intestine and is sutured in place on the mesentery.

FIG. 10 is a front, internal view of a human thoracic cavity with the lungs therein and pericardial cavity therein outlined by a dashed line.

FIG. 11 is a cross-sectional, close-up view of the lung and chest wall shown in FIG. 10 with an adhesion. The adhesion is extended and somewhat exaggerated to show what it would look like prior to division.

FIG. 12 is a cross-sectional view of the lung, adjacent chest wall, and the adhesion as shown in FIG. 1 after division of the adhesion.

FIG. 13 is a cross-sectional view of the lung, adjacent chest wall and divided adhesion shown in FIG. 12 after application of the present invention composition of cells and protein to the injured surfaces of the lung and chest wall.

FIG. 14 is a cross-sectional view of the lung, adjacent chest wall, divided adhesion, and covering layer of cells and protein shown in FIG. 13, where an optional layer of tissue sealant or tissue adhesive is applied to temporarily stabilize the composition.

### DETAILED DESCRIPTION OF THE INVENTION

A preferred embodiment of the present invention comprises harvested and/or cultured cells suspended in fibrin glue and applied to an injured surface. Alternatively, other proteins, polysaccharides or polymers may be used. The suspension of viable cells in the protein, polysaccharide or glue assures rapid reformation of the epithelial layer by the deposition of these cells on the surface of the injury as the composition is absorbed. Cells suspended in a fast absorbing protein or polysaccharide or glue ideally allows for one application of the composition to achieve separation and a source of seed cells. Seeding will occur as the protein is absorbed, maximizing the ease of use of the present invention. This is particularly important for endoscopic procedures where it is often time consuming to apply a coating or a strip of mesh or fabric.

The preferred embodiment may be used in situations where a limited number of cells are available to rapidly reform the epithelial layer, such as major surgery or trauma to an internal organ or cavity wall. Not being bound by theory, it is thought that absorbance of the protein, polysaccharide or glue occurs through enzymatic action at the surfaces of the composition releasing nourishment to the cells and allowing macrophages to naturally consume the non-viable material. This may also permit migration of cells along the injured surface under the composition layer. This enzymatic action also occurs on both surfaces of the composition layer such that some cells are lost in the body cavity, but it is normal for such cells to migrate within body cavities such as in the peritoneal cavity. As deposition of the cells occurs, and the seeded cells grow, a layer of viable cells is formed. With adequate nourishment, the seed cells will quickly grow to form a new epithelial layer without adhesions. Since scar tissue forms very rapidly, adhesion prevention methods should be used during the early stages of healing. Therefore, fast absorption and formation of new cells is important. Under normal conditions, a thin covering of epithelial cells can form in about three days.

Preferably the protein is a fibrin glue, but it may also be an absorbable tissue sealant or an adhesive. The fibrin glue is a two-part liquid that is blended prior to contacting the surface. The viable cells are mixed with the fibrin glue, and the glue is then polymerized. Cells may be added to one or both of the two-part liquids.

The sealant or glue used in the composition may be selected from a number of sources or types. Preferably the sealant, glue or adhesive is comprised of a recombinant human plasma protein as a main component. Such sealants, glues or adhesives contain a crosslinking composition, which may comprise an aldehyde (see U.S. Pat. No. 6,329,337, Morita, issued Dec. 11, 2001, which is herein incorporated by reference), collagen, albumin or fibrin as a main component (see U.S. Pat. Nos. 5,786,421, Rhee et al., issued Jul. 28, 1998, and 5,583,114, Barrows et al., issued Dec. 10, 1996, which are herein incorporated by reference). An example of a protein tissue sealant is Tisseel VH fibrin sealant, manufactured by Baxter Health Care Division of Baxter International Inc. Other fibrin glue manufacturers include Centeon, Marburg, Germany; Bio-transfusion, Lille, France; Nycomed Pharma, Roskilde, Denmark; and Haemacure Inc., Quebec, Canada. Fibrin glue is thought to replicate the last stages of the natural hemostasis cascade (or polymerization) of fibrinogen into fibrin monomers followed by cross-linking into a fibrin matrix. Other proteins, which do not constitute glues or adhesives, that may be used include Gelatin Sponges, FloSeal Matrix Hemostatic Sealant, and collagen-derived particles and topical thrombin. In most of these cases, diluting the protein, sealant or glue is desirable in order to increase the rate of absorbance and reduce the acidity from enzymatic action.

Autologous fibrin glue can be made by centrifuging the patient's own blood and removing the supernate, which is plasma that contains fibrinogen. The plasma, when combined with thrombin and calcium, will form fibrin very quickly and should be applied to the wound as the mixing is occurring. Thrombin is commercially available from Fusion Medical Technologies, a division of Baxter International Inc. To form the composition of the present invention, cells may be suspended in the plasma or thrombin or both. The fibrinogen concentration is diluted in the plasma and may limit the adhesive strength when making autologous fibrin glue. When using the composition on mobile organs such as bowel or lung, the fibrinogen concentration may be increased by two freezing cycles at about −18° C. or by using ammonium sulfate or ethanol to precipitate out the fibrinogen. Diluted fibrin glue is preferred because it absorbs quickly. If greater adherence or stability is required, a second and broader coat of concentrated glue may be applied over the diluted glue/cell suspension.

In accordance with the principles of the present invention, an element may be added to the composition to ensure that it is stabilized and remains in place to separate the surfaces during movement of an organ, such as during peristalsis (bowel) or respiration (lungs). Stabilization refers to keeping the composition of seed cells and protein or polymer in place during the healing process. The need for a separating and stabilizing element is related to the stability of the injured organ or body cavity wall. A tissue sealant, adhesive or an absorbable strip, mesh or fabric may be used to cover and stabilize the composition such that it remains in place.

Stabilization is less important in situations where the organs remain relatively free of motion such as in the pelvic cavity. Gels or pastes are often adequate for this type of organ and the surrounding body cavity wall. However, in the case of bowel and lungs, some type of stabilizing element is required since the organs are constantly in motion. In the case of the lung, it is difficult to suture a mesh or fabric in place, so a tissue adhesive or sealant is preferred. In addition, a strip is not easily attached to the abdominal or chest wall. However, stabilization of the composition layer on the chest or abdominal wall may not be required since relative motion is less and the blood supply is significantly better which will promote faster healing. It is preferable to use tissue adhesives or sealants to secure the composition to the abdominal or chest wall if required, or to function as both a stabilizing element and to suspend/sustain cells for delivery of viable epithelial cells to the injured surface. When a mesh is used, it can be relatively thin and low density since it only needs to remain in the patient for about three days. The density of said mesh, fabric, or strip is preferably less than about 8 mg/cm² to reduce the acidity resulting from enzymatic action upon absorption.

The said mesh, fabric, or strip preferably has open areas or pores to allow grafted cells to migrate through to the surface of the injury. This allows the composition of protein, glue, or polysaccharide and suspended viable cells to be applied over the mesh or fabric or applied both under and over the mesh or fabric. Application of the suspension portion of the composition is preferred to be done after the mesh or strip is in place to avoid the potential of wiping away or dislodging the suspension composition when attempting to install a mesh, fabric, or strip. The stabilizing element may be fabricated from a rapid absorbing material such as a lactide and/or glycolide polymer or copolymer. The irradiated (or Rapide) version of such polymers would be preferred since they have faster absorption. Another alternative is an oxidized, regenerated cellulose fabric or mesh with a density preferably less than about 8 mg/cm².

A preferred embodiment for practicing the method of the present invention comprises the steps of:

(a) harvesting and, if needed, culturing autologous, non-keratinizing, epithelial cells and suspending said cells in a thin fibrin glue to make a composition of the present invention (alternatively a protein or polysaccharide could be used in place of the fibrin glue where stability is less important);

(b) surgically accessing a portion of the human bowel, abdominal wall, lung, pleura, or other such structures having an adhesion, via an incision or other means;

(c) assuring the region surrounding the injury is free of infection and contamination;

(d) applying said composition of the present invention to the injured surface;

(e) where required, applying an absorbable tissue adhesive or a flexible strip, mesh, or fabric to cover said composition as a stabilizing and separating element and if required securing said stabilizing element with sutures or tissue adhesive; and

(f) closing the access incision(s).

Alternatively, since said stabilizing element (strip, mesh, or fabric) is porous, said composition may be applied before, after, or both before and after application of said strip used for stabilization. The mesh or fabric need not be of a certain density but preferably has a density of less than about 8 mg/cm².

In accordance with a further embodiment of the present invention, when the injury is small, such as the division of an existing adhesion or minor injury to the surface from manipulation of tissue, the application of protein fibrin glue may be all that is necessary. Alternatively, with applications for larger injuries, cells may be seeded directly onto the injured surface prior to application of protein or fibrin glue. The direct seeding method is practical for open procedures where access is improved or where a large number of cells are available.

In accordance with the principles of another embodiment of the present invention, said composition may also be used to deliver medications, growth factors or nutrients to the injured surface. For example, fibronectin is a growth factor valuable in the inducement of epithelial development that could be delivered directly to the injured surface to increase the probability of successful grafting or healing without adhesion formation.

Epithelial cells used in accordance with the principles of the present invention should be harvested or derived from non-keratinizing surfaces or in other words from internal, non-epidermal epithelial surfaces. Keratin is a protein formed by keratinizing cells in the dermis. External dermal cells (i.e., skin cells) are keratinizing and will grow in the internal environment and can pose a threat of malignancy. Sources of harvested cells include cadavers, donors or autologous cells. To avoid the potential of rejection, autologous cells are preferred. These harvested cells can be cultured to provide adequate amounts for large wounds.

Cells may be harvested from the mouth or from other internal epithelial sources such as from the adhesions that are divided or harvested. The mouth provides the easiest access and the cells are identical to those found in the abdominal and thoracic cavities. Harvesting can be achieved by simply scraping cells from the inside of the cheek with a sterile spatula. Care must be taken to assure that the cells are aseptic by cleaning the harvested site in advance and washing and centrifuging the cells. The cells may also be washed and centrifuged to assure they are clean and free of debris. Other sources of cells may be from the adjacent epithelial layer or the cells may be present on the injury surface. Harvested cells are prepared by separating them prior to suspension in the protein. This may be achieved by treating the cells with a 0.25% ethylenediamineteteracetic acid (EDTA) solution for approximately 45 minutes at about 37° C., in order to separate into individual cells. The solution may be centrifuged to yield concentrated individual cells.

The protein cell suspension composition may be delivered to the injured surface using a number of methods appropriate for the type of surgery and the protein selected. For most proteins, a syringe may be the best method for delivery in both open and endoscopic surgery. For endoscopic surgery, a long needle or tube may be required. However, for fibrin glue and tissue sealants, a method that mixes the two-part liquids as they are applied to the injury is required. Such devices are available from fibrin glue and tissue sealant manufacturers. A simple aerosolization device or sprayer that will simultaneously spray the two-part liquids onto the surface is the preferred method. The seed cells can be in suspension in one or both liquids or be applied in advance or simultaneously in a separate suspension composition.

Another source of seed cells are the cells that were not destroyed or removed when the injury occurred. Alternatively, the injury may be small enough that cells surrounding the injury may be adequate to reform an epithelial layer. This would be true for small injuries such as when a small adhesion is divided or cut. Also, one of the novelties of the current invention is the capability for cells to migrate under the cover of the composition layer as it is absorbed. Therefore, an adhesion may be treated by dividing it and applying the composition of the present invention without additional grafted or suspended cells.

### EXAMPLE

Six female patients, with a history of abdominal surgery and chronic pain are admitted for exploratory laparoscopic surgery to diagnose the reason for the pain. The pain is found to be due to omental and/or bowel adhesions. The adhesions are divided and injuries resulting from the division are treated with a layer of fibrin glue. From 4 to 10 months postoperative, the patients are laparoscopically re-evaluated, with 5 of the 6 patients found to be free of adhesions. It is also observed that the one patient who continues to have adhesions did not originally have a proper division along epithelial planes. The division is observed to have been into the abdominal wall rather than separating epithelial planes. Since the division was made in a space that had no epithelium, there were no epithelial cells existing on the injured surfaces to seed the reformation of the epithelium. Once the fibrin glue was absorbed, the space closed by healing, and the condition reoccurred with a reoccurrence of the pain. Had there been epithelial cells present reoccurrence could have been avoided.

This example demonstrates that the epithelial layer can reform from surrounding seed cells and the protein sealant may nourish the seed cells. However, the number of cells available to seed the injured area may not be adequate for major injuries. Surgically cutting existing adhesions creates minor injuries in comparison to the trauma of a surgery such as lung or bowel resection. Larger areas of injury will require harvesting epithelial cells for grafting or seeding of the injured area to supplement the sources of seeding discussed above in the creation of an epithelial layer.

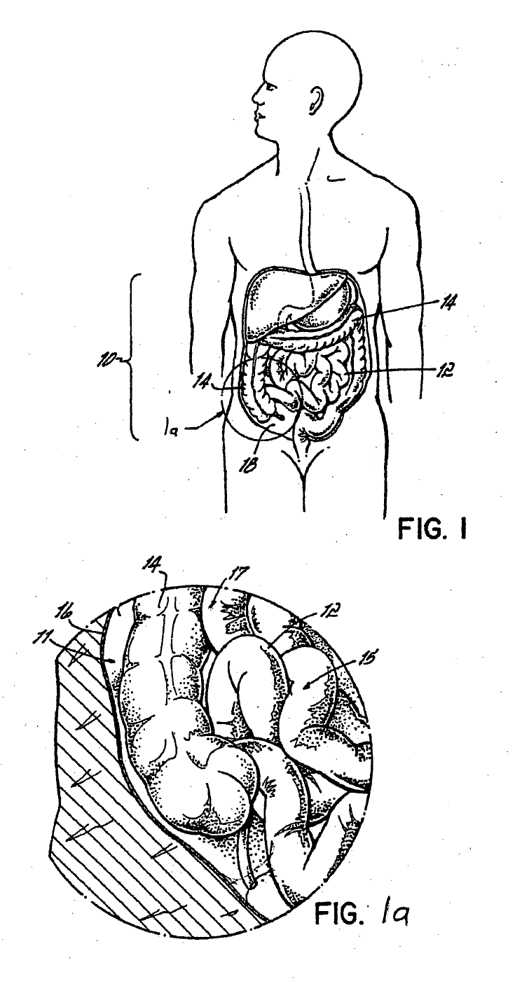

As shown in FIGS. 1 and 1a, a person's internal mid-section 10 is occupied by the small intestine 12 (which has a number of undulating loops 15, 17) and the large intestine 14, collectively and more generally called the bowel. Both small intestine 12 and large intestine 14 are completely encased by the peritoneum 16 and located within the peritoneal cavity 11. Small intestine 12 and large intestine 14, are lubricated by peritoneal fluid and this allows the small intestine 12 and large intestine 14 to move freely within cavity 11. Mobility is critical to proper functioning of both intestines 12, 14. During the process of digestion, food is moved along intestines 12, 14 by involuntary waves of contractions, better known as peristalsis. Loss of such mobility results in loss of proper functioning of intestines 12, 14 that in turn, may prove very painful or even fatal.

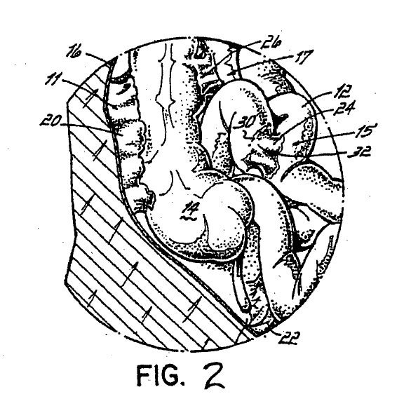

As shown in FIG. 2, loss of mobility may be the result of peritoneal adhesions 20, 22, and/or bowel to bowel (or organ to organ) adhesions 24, 26. Peritoneal adhesions 20, 22 form between the peritoneum 16 and intestines 12, 14. Bowel to bowel adhesions 24, 26 form between opposing surfaces 30, 32 of the same organ, such as small intestine 12. Inter-organ adhesions 26 form between adjacent organs, such as small intestine 12 and large intestine 14. Adhesions 20, 22, 24, 26 may result from trauma sustained by peritoneum 16 or by intestine 12, 14. Adhesions 20, 22, 24, 26 may organize into permanent adhesions by incorporating collagen. The formation of permanent adhesions is usually accompanied by pain and loss of intestinal mobility and function.

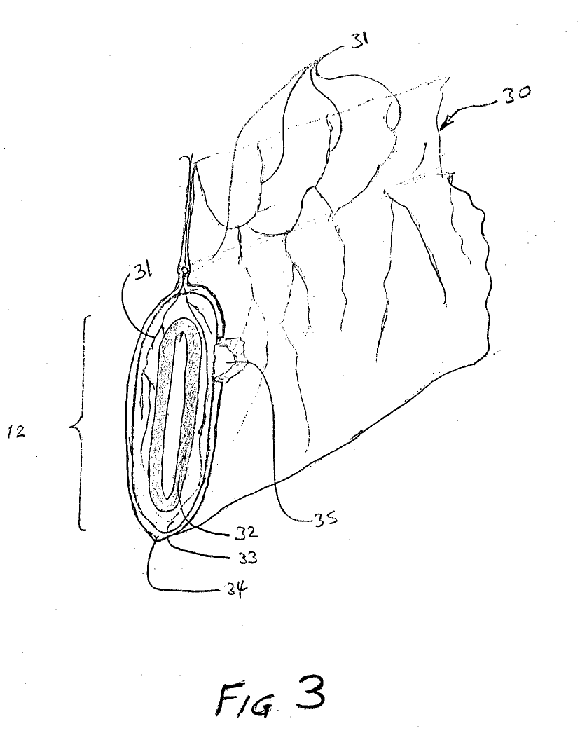

FIGS. 3-9, show a method of treating adhesions of the small intestine 12, which can also be used to treat the large intestines 14 (FIGS. 1 and 1a) or other organs such as urinary bladder and sigmoid colon in the human pelvis, lung in the thoracic cavity, and the mediastinal organs such as pericardium, spinal cord, dura, tendon or tendon sheath in accordance with the principles of the present invention. The illustrations and teaching of the preferred embodiment of the present invention may be used for both treatment and prevention of adhesions. For purposes of illustration, the treatment and methods described herein may be applied to the prevention of adhesions where injury is caused by surgery or trauma. FIG. 3 shows the intestine 12, which has a mesentery ligament 30 with blood supply 31. The intestine 12 has mucosal layer 32, muscle layer 33 with blood supply 31, and serosal layer or visceral peritoneum 34. The serosal layer is an epithelium and consists primarily of non-keratinizing or non-epidermal, epithelial cells. The peritoneum 34 has an injury 35 that was created when an adhesion was divided or cut which leaves a deficit or void in the peritoneum.

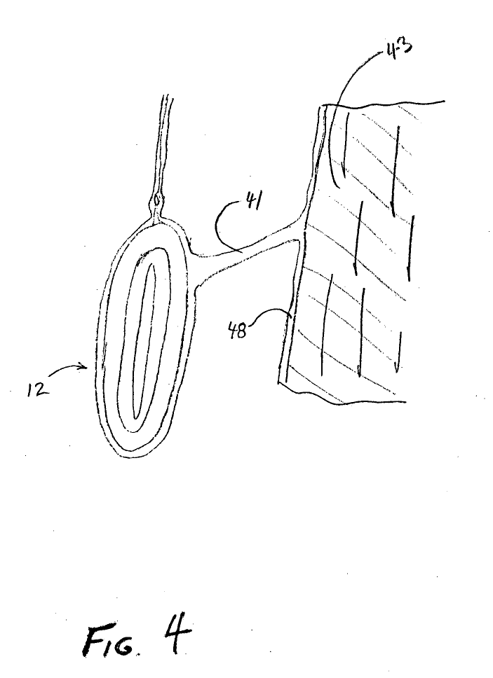

With reference to FIG. 4, in order to conduct a surgical procedure, the intestine 12 and abdominal wall 43 with parietal peritoneum 48 must first be surgically accessed to expose and extend adhesion 41 which will represent a treatment of the present invention that could also be applied to other adhesions. Once the desired portion of intestine 12 has been exposed, the adhesion 41 is divided or freed from intestine 12, which is exemplary of adhesions 20, 22, 24, 26 in FIG. 2. The division of intestines 12 can be achieved in various ways such as will be readily appreciated by those skilled in the art. One way to free intestines 12 is by division with a sharp dissecting instrument or surgical scissors to allow complete access to the injured intestinal surfaces.

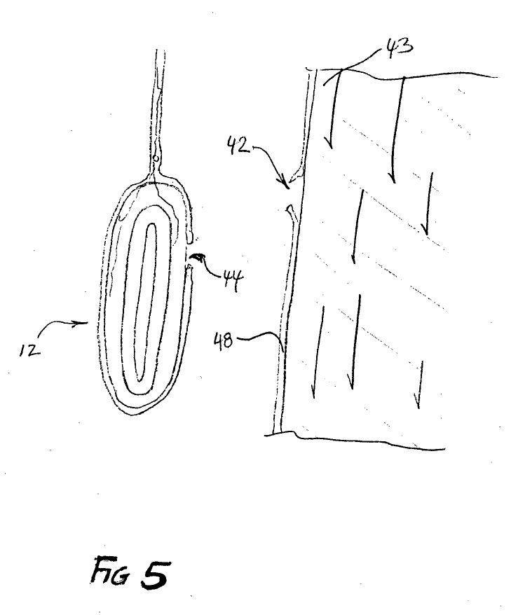

With reference to FIG. 5, division of the adhesion 41 creates an injury 42 in parietal peritoneum 48 on abdominal wall 43 and a similar injury 44 on the intestine 12. Such injuries, as well as injuries for other surgical procedures or from manipulation of the tissue, can lead to occurrence or in this case reoccurrence of adhesion(s). Such injury causes formation of scar tissue if intestine 12 were to be in contact with abdominal wall 43, other portions of the intestine 12, or other internal organs. Contact is highly likely since it is normal for the visceral (internal organs) surfaces to be in direct contact. The two surfaces adhere and grow together creating an adhesion(s).

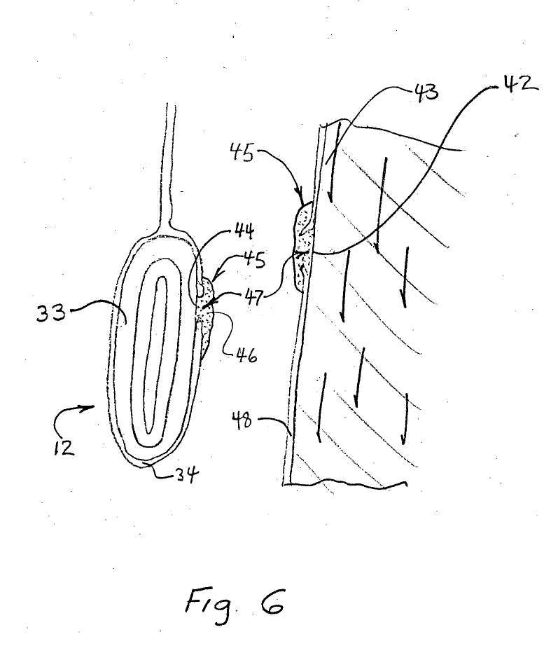

In accordance with the a preferred embodiment of the present invention, non-keratinizing epithelial cells are harvested and mixed with a protein liquid, paste, or gel to yield a composition. Preferably the protein is fibrin glue but may also be a tissue sealant or adhesive. The viable cells are mixed with the fibrin glue, which is a two-part liquid that is blended as it is applied to the surface, allowing polymerization of the glue to start as the glue is being applied. Polymerization is generally completed after application to the surface to yield the composition 45 shown in FIG. 6. Cells may be added to one or both of the two-part liquids.

FIG. 6 shows injuries 44, 42, covered by the composition of the present invention 45 that contains harvested epithelial cells (examples 47 indicated) suspended in a protein 46, and is preferably fibrin glue. The composition 45 insures that adequate epithelial cells and nutrition is available for the reformation of the peritoneum 34, 48, and separates the injured surfaces or separates the injury from other surfaces to avoid the reformation of adhesions.

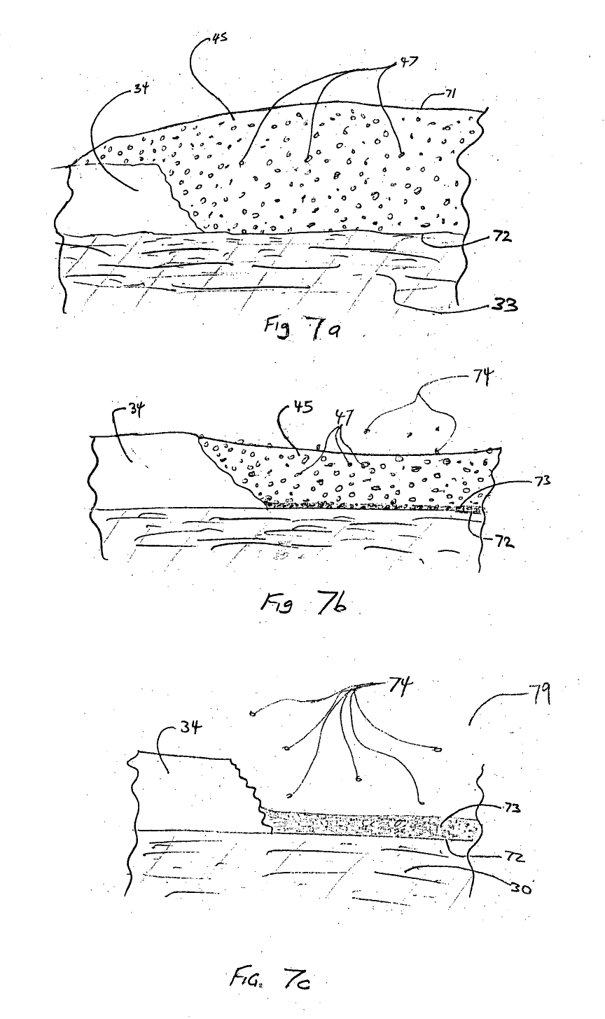

FIGS. 7a, b and c illustrate how the composition 45 supplies an adequate amount of viable seed cells 47 for reformation of the epithelial layer, which in the case of the small intestine, is the visceral peritoneum 34. Composition 45 is covering the injuries and extends or overlaps with the uninjured surface of the peritoneum 34 and covers the muscle layer 33 of the intestine 12 at its surface 72. FIG. 7b shows seed cells forming a thin epithelial layer 73. The layer 73 is formed by the deposition of the viable cells 47 as the composition is absorbed through enzymatic action at the surface 72 releasing nourishment to the cell layer 73. Excess nourishment will be consumed by macrophages in the muscle layer 30. This enzymatic action also occurs on the exposed surface 71 of the composition 45 such that some cells are lost in to the body cavity or space surrounding the injury. Such cells become free cells 74 which also may be present naturally in the body cavity, and such free cells 74 are a source of viable cells for seeding the injury 35. FIG. 7c show that with adequate nourishment, the cell layer 73 will grow to form a new epithelial layer to reform the peritoneum 34 without adhesions. The composition 45 has been absorbed exposing the cell layer 73 covering muscle layer 30 such that further healing will not form an adhesion now that the injury is not separated from other surfaces by the composition 45.

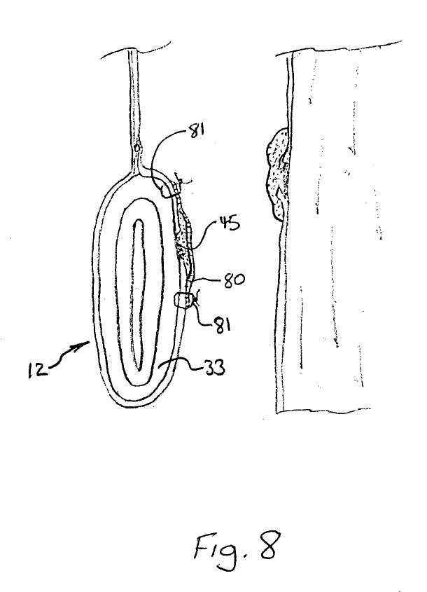

In accordance with the principles of the present invention, FIG. 8 illustrates a method to assure that composition 45 is stabilized and remains in place during peristalsis. The injured area 44 and composition 45 are covered with an absorbable mesh or fabric 80. The absorbable mesh or fabric is secured to the intestine 12 by sutures 81. The sutures are placed in the muscle layer 33 without puncturing through the intestine or mesentery. Added separation and improved stability further reduces the chance of any adhesions forming during the healing process.

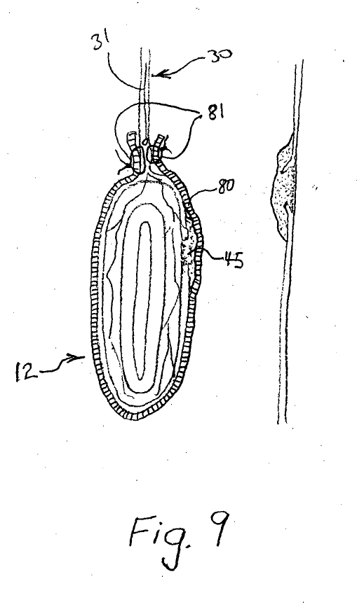

To further avoid potential penetration of the intestinal wall, FIG. 9 illustrates that the absorbable mesh or fabric 80 may be placed around the intestine 12 and sutured to the mesentery 30. Some care must be taken in this method to avoid the blood supply 31 in the mesentery 30. Typically, suturing mesh or fabric 80 to intestines 12 will maintain mesh or fabric 80 on intestine 12 long enough to allow composition 45 to be absorbed and/or reform a thin layer of epithelial cells. Any leakage from intestine 12 is undesirable as such leakage creates an environment conducive to the growth of bacteria and subsequent infection. If an infection results at the site of composition 45 or the absorbable mesh or fabric 80 on intestine 12, the infection may consume the composition, mesh, or fabric, using it as an energy source. If the composition 45 is consumed and therefore not present on intestine 12, adhesions will likely form and further complications will result from the infection. Infection is a threat to the success of the treatment, but the threat can be reduced by using irrigation with sterile and medicated saline solution to wash out contamination that may lead to infection. Irrigation methods and solutions will be readily appreciated by those skilled in the art.

Alternatively, viable cells 47 may be omitted from the composition 45 if the injured surface 44 is small and the serosal layer 33 is sufficiently intact to provide seed cells. The methods are the same as those illustrated in FIGS. 3-9 except that viable cells 47 are omitted from the composition. Viable cells are available at the edges of the injury 44, and some may remain on the injured surface, or remnants may be present deep in the injury. Experience has shown this embodiment of the present invention to be adequate for reformation of the epithelial layer in time to prevent adhesions but only for a very minor or small injury.

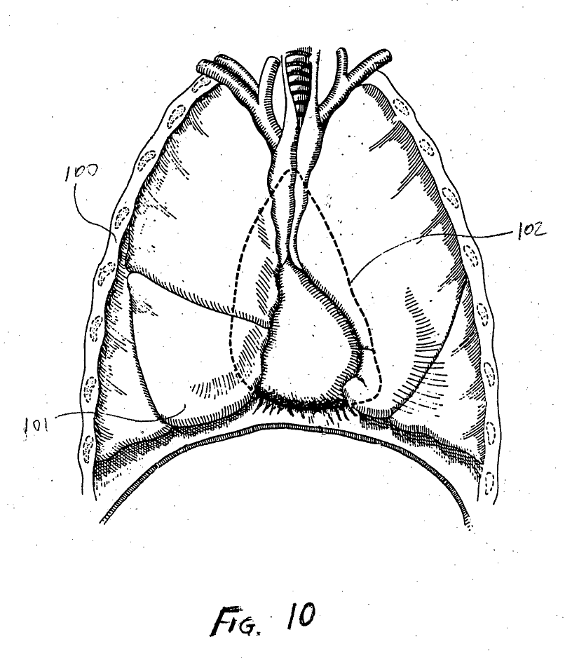

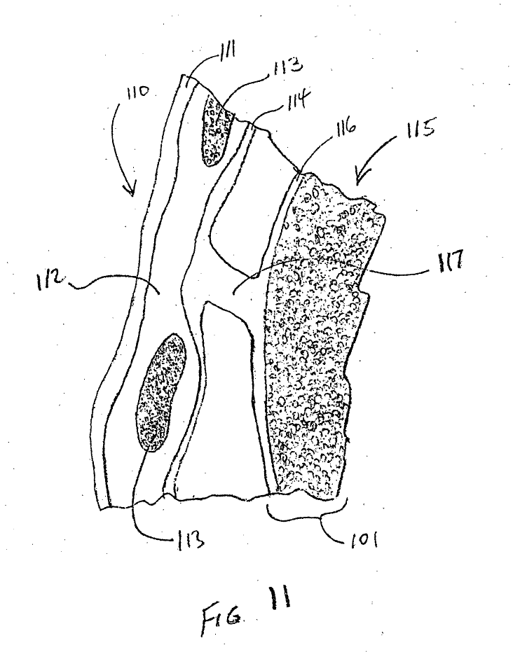

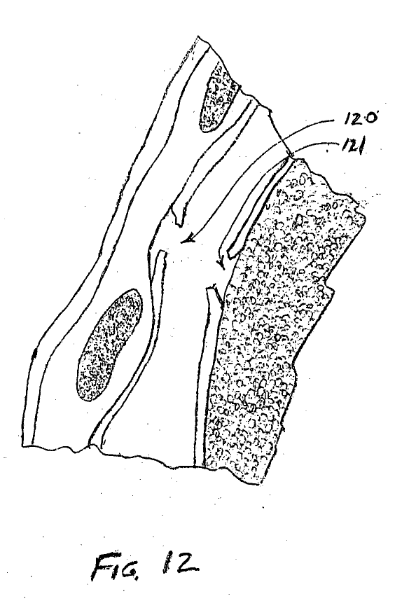

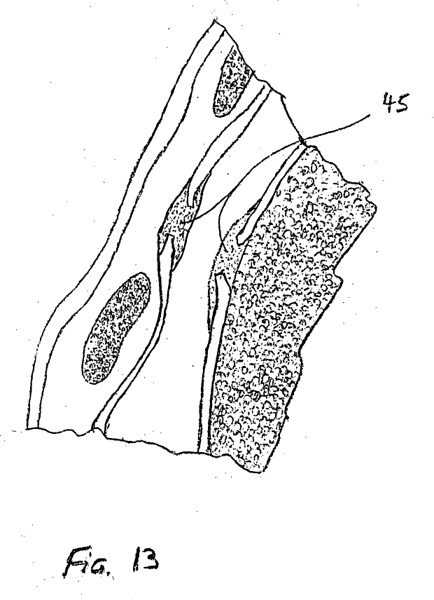

In accordance with the principles of the present invention, FIGS. 10-14 exemplify the invention as applied to the thoracic cavity. The preferred embodiment and other embodiments may be applied to other body cavities such as the pelvis, pericardium, spinal cord, dura, tendon or tendon sheath. Using the composition and methods described above, FIG. 10 shows the human thoracic cavity with chest wall 100, lungs 101 and pericardial cavity 102. The cavity may be accessed via an incision (not shown) in the chest wall. FIG. 11 is a close-up view of a cross-section of the chest wall 110 comprising the skin 111, muscle layer 112, ribs 113 and parietal pleura 114. The lung 101 is shown with parenchyma 115 and visceral pleura 116 covering the lung. The visceral pleura 116 is an epithelial layer of the lung. Adhesion 117 has formed from a previous surgery or disease and is immobilizing the lung 101. Adhesion 117 is divided using a method known to those skilled in the art. The divided adhesion 117 which resulted in injury 120 and 121 shown in FIG. 12.

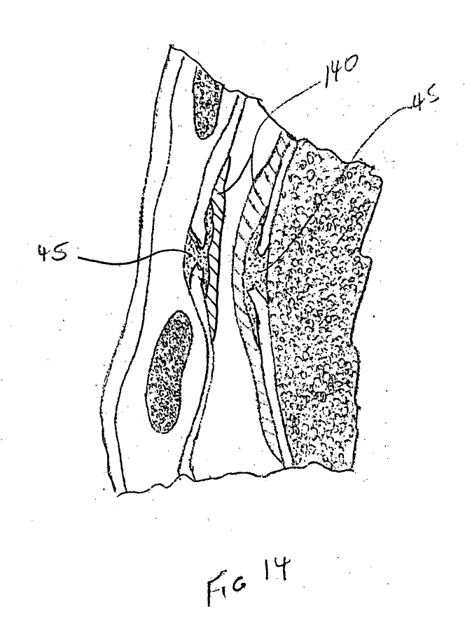

Alternatively, viable cells may be seeded in the injured area and the fibrin glue applied over the seed cells. If sealing and adherence is adequate, no further stabilization or separation is needed. FIG. 14 shows the application of a second layer of adhesive or sealant 140 over the composition 45 containing viable cells, which stabilizes the composition 45 and adds another layer of separation. This added layer 140 allows the use of a liquid, paste or gel protein to form the composition 45 where the paste or gel is secured to the lung by the added layer of adhesive or sealant 140.

By virtue of the foregoing composition and methods, most adhesions maybe prevented or treated with little or no re-occurrence. While the present invention has been described by several examples, it is not the intention of Applicant to restrict or in any way limit the scope of the invention to those skilled in the art. For example, various proteins and polymers can be used to suspend viable cells, and faster and easier methods for harvesting and applying cells to the injured surface may also be utilized.

What is claimed is:

1. A composition for the prevention or treatment of adhesions adapted for application to an injured internal surface of the body comprising:
a. viable, non-epidermal, epithelial cells;
b. an absorbable substance capable of maintaining the viability of said cells; and
c. a means to temporarily separate the injured surface from surrounding tissue surfaces;
wherein said absorbable substance at least partially suspends or covers said epithelial cells.

2. The composition of claim 1 wherein said absorbable substance is the means or comprises a portion of the means to temporarily separate the injured area from other tissue surfaces.

3. The composition of claim 1 wherein said absorbable substance comprises a material selected from proteins, polysaccharides, hyaluronic acid, hyaluronate (polysaccharide), collagen, albumin, tissue sealants, fibrin-based tissue sealants, fibrinogen, polymers, polymerizable monomers, cyanoacrylates, and mixtures thereof.

4. The composition of claim 1 wherein said absorbable substance provides nutrition for said epithelial cells.

5. The composition of claim 1 wherein said absorbable substance is selected from a protein-based tissue sealant, glue, or adhesive that polymerizes, dries, or forms a stabilizing layer without activation by a radiant energy stimulus, and mixtures thereof.

6. The composition of claim 1 wherein said means to temporarily separate the injured area comprises a mesh, fabric, or strip of polymer which carries said absorbable substance and said epithelial cells.

7. The composition of claim 6 wherein said mesh, fabric, or strip comprises an absorbable polymer material selected from oxidized regenerated cellulose, polymers or copolymer of glycolic acid, lactic acid and related monomers, polydioxanone, polytrimethylene carbonate, polyalkylene glycol, polycaprolactone, hyaluronic acid, hyaluronate, cyanoacrylates, and mixtures thereof.

8. The composition of claim 6 wherein said mesh, fabric, or strip is sufficiently porous to allow suspended cells, implanted cells, or surrounding free cells to migrate and graft onto the injured area.

9. The composition of claim 6 wherein said mesh, fabric, or strip has a density of less than about 8 mg/cm².

10. The composition of claim 6 wherein said absorbable substance is applied over one or both sides of said mesh, fabric or strip.

11. The composition of claim 1 wherein said epithelial cells are harvested from autologous blood or tissue or cultured from cells harvested from the patient to be treated.

12. The composition of claim 11 wherein said epithelial cells are harvested from the patient's mouth or from surfaces comprising non-keratinizing cells.

13. The composition of claim 1 wherein said absorbable substance is a layer or layers comprising a material selected from coarse granules, fibers, and/or mesh, and where said layer or layers are adapted to adhere to the injured surface by an adhesive, or by the wetness of the tissue.

14. The composition of claim 3 wherein said absorbable substance is fibrin glue.

15. The composition of claim 1 in the form selected from a liquid, paste, gel, solid, mesh or fabric.

16. A method for the prevention or treatment of adhesions comprising applying a composition comprising fibrin-based tissue sealant, glue, or adhesive to one or more injured internal surfaces so as to temporarily separate the injured area from other tissue and to protect, nourish and promote the regeneration of epithelial cells for reformation of an epithelial layer over said injured area.

17. The method of claim 16 wherein the composition includes viable, non-epidermal, epithelial cells.

18. The method of claim 17 wherein the composition sustains the viability of the epithelial cells.

19. The method of claim 18 wherein the injury occurred in the course of a surgical procedure.

20. The method of claim 19 wherein the surgery is performed in the pelvis, abdomen, thoracic cavity, pericardium, spinal cord, dura, tendon or tendon sheath cavity.

21. A method for the treatment and prevention of adhesions in a patient, comprising the steps of:
a. surgically accessing an animal or human pelvis, abdomen, thorax, pericardium, spinal cord, dura, tendon, tendon sheath, or tissues covered by an epithelial layer where adhesion(s) have formed or may form;
b. dividing one or more adhesions that may be present or conducting other surgery, thereby forming an injured area;
c. providing viable epithelial cells or assuring that viable epithelial cells are present in or surrounding said injured area; and
d. applying an absorbable substance, protein or polymer in one or more layers over said viable epithelial cells and said injured area to stabilize and temporarily separate the injured area from surrounding organ surfaces.

22. The method of claim 21 wherein said absorbable substance polymerizes, dries, or forms a stabilizing layer without activation by a radiant stimulus.

23. The method of claim 21 wherein all or a portion of said epithelial cells are suspended in said absorbable substance, protein, or polymer for delivery to the injured area.

24. The method of claim 21 further comprising applying one or more strips of absorbable sheet, mesh or fabric over said injured area, said absorbable substance layers being applied under and/or on top of said strips.

25. The method of claim 24 wherein said strips of absorbable sheet, mesh or fabric have a density of less than about 8 mg/cm² and are permeable by free epithelial cells.

26. The method of claim 25 further comprising attaching of said absorbable sheet, mesh or fabric to the injured area.

27. The method of claim 26 wherein said attachment is achieved by suturing, clipping, stapling, or using a glue, adhesive or sealant for the purpose of bonding said strips in place.

28. The method of claim 21 wherein said viable epithelial cells are harvested from autologous tissue or cultured from cells harvested from the patient.

29. The method of claim 28 wherein said viable epithelial cells are harvested from the patient's mouth or from surfaces comprising non-keratinizing cells.

30. The method of claim 21 wherein at least a portion of the absorbable substance is derived from an animal, cadaver, human donor blood or tissue, or autologous blood or tissue.

31. The method of claim 21 wherein said viable cells are epithelial cells surrounding or present in said injured area.

32. The method of claim 21 wherein said absorbable substance is also used for delivery of medications, growth factors or nutrients to said injured area.

33. The method of claim 21 wherein the absorbable substance comprises fibrin glue.
